SEQUENCE LISTING (44 sequences)

SEQ ID NO: 1 - DNA, 2859 bp, human
ctttccgcggcattctttgggcgtgagtcatgcaggtttgcagccagccccaaagggggtgtgtgcgcgagcagagcgctataaatacggcgcctcccagtgcccacaacgcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcaaaaaaaaaaaaaaaaaa

SEQ ID NO: 2 - DNA, 21 bp, human
gcacagcaggagaatcttcat

SEQ ID NO: 3 - DNA, 21 bp, human
tggagtctttgcacgcctcgg

SEQ ID NO: 4 - DNA, 21 bp, human
cagcagcagagtcttcatcat

SEQ ID NO: 5 - DNA, 21 bp, human
attgtctgagaccgtctggtc

SEQ ID NO: 6 - DNA, 21 bp, human
ccttcagctttgtctctgatt

SEQ ID NO: 7 - DNA, 21 bp, human
agcagggagtcgatgcggtca

SEQ ID NO: 8 - DNA, 21 bp, human
atcaagctgcggacgatgcgg

SEQ ID NO: 9 - DNA, 21 bp, human
gcaggcagcccgtggagttgt

SEQ ID NO: 10 - DNA, 21 bp, human
ttcagctgctccagcaaggag

SEQ ID NO: 11 - DNA, 21 bp, human
aatttagggttcttcctggag

SEQ ID NO: 12 - DNA, 21 bp, human
gctgggcggagttgggggcct

SEQ ID NO: 13 - DNA, 17 bp, human
ggtgtagacgccgcacg

SEQ ID NO: 14 - DNA, 16 bp, human
gcagcgcagcccctgg

SEQ ID NO: 15 - DNA, 22 bp, human
gcagcagccgcagcccggctcc

SEQ ID NO: 16 - DNA, 18 bp, human
agccgcagcccggctcct

SEQ ID NO: 17 - DNA, 20 bp, human
cagcagccgcagcccggctc

SEQ ID NO: 18 - DNA, 20 bp, human
gcagcagccgcagcccggct

SEQ ID NO: 19 - DNA, 20 bp, human
agcagccgcagcccggctcc

SEQ ID NO: 20 - DNA, 21 bp, artificial
  mismatch primer
cagcagcagagtatttatcat

SEQ ID NO: 21 - DNA, 21 bp, human
guagaagggcgagcucuggtt

SEQ ID NO: 22 - DNA, 21 bp, human
gaugcucaacaccuccucctt

SEQ ID NO: 23 - DNA, 21 bp, human
ggaggagguguugagcauctt

SEQ ID NO: 24 - DNA, 21 bp, human
cuaauucaauaaaacuguctt

SEQ ID NO: 25 - DNA, 21 bp, human
gacaguuuuauugaauuagtt

SEQ ID NO: 26 - DNA, 19 bp, human
uaauucaacaaaacugutt

SEQ ID NO: 27 - DNA, 19 bp, human
acaguuuuguugaauuatt

SEQ ID NO: 28 - DNA, 21 bp, human
augaugaagacucugcugctt

SEQ ID NO: 29 - DNA, 21 bp, human
gcagcagagucuucaucautt

SEQ ID NO: 30 - DNA, 22 bp, human
ugaaugaagggacuaaccugtt

SEQ ID NO: 31 - DNA, 22 bp, human
cagguuagucccuucauucatt

SEQ ID NO: 32 - DNA, 22 bp, human
cagaaauagacaaaguggggtt

SEQ ID NO: 33 - DNA, 22 bp, human
ccccacuuugucuauuucugtt

SEQ ID NO: 34 - DNA, 22 bp, human
acagagacuaagggaccagatt

SEQ ID NO: 35 - DNA, 22 bp, human
acagagacuaagggaccagatt

SEQ ID NO: 36 - DNA, 21 bp, human
ccagagcucgcccuucuactt

SEQ ID NO: 37 - DNA, 21 bp, human
guagaagggcgagcucuggtt

SEQ ID NO: 38 - DNA, 21 bp, human
gucccgcaucguccgcagctt

SEQ ID NO: 39 - DNA, 21 bp, human
gcugcggacgaugcgggactt

SEQ ID NO: 40 - DNA, 21 bp, human
cuaauucaauaaaacuguctt

SEQ ID NO: 41 - DNA, 21 bp, human
gacaguuuuauugaauuagtt

SEQ ID NO: 42 - DNA, 19 bp, human
augaugaagacucugcugc

SEQ ID NO: 43 - DNA, 19 bp, human
gcagcagagucuucaucau

SEQ ID NO: 44 - DNA, 21 bp, human
ccagagcucgcccuucuactt

(19) United States
(12) Patent Application Publication
(10) Pub. No.: US 2011/0142827 A1
(43) Pub. Date: Jun. 16, 2011

(54) TREATMENT OF CANCER WITH A COMBINATION OF AN AGENT THAT PERTURBS THE EGF SIGNALING PATHWAY AND AN OLIGONUCLEOTIDE THAT REDUCES CLUSTERIN LEVELS

(75) Inventors: Martin Gleave, Vancouver (CA); Gabriella Zupi, Roma (IT)

(73) Assignee: The University of British Columbia

(21) Appl. No.: 12/886,027

(22) Filed: Sep. 20, 2010

Related U.S. Application Data
(63) Continuation of application No. 11/718,815, filed on May 18, 2007, now abandoned.
(60) Provisional application No. 60/522,948, filed on Nov. 23, 2004.
(60) Provisional application No. 60/522,960, filed on Nov. 24, 2004.

(51) Int. Cl. A61K 39/395; A61P 35/00

(52) U.S. Cl. 424/133.1

(57) ABSTRACT

Agents that perturb the EGF signaling pathway and that are known to be useful in the treatment of cancer are found also to result in increased expression of the protein clusterin. Since clusterin can provide protection against apoptosis, this secondary effect detracts from the efficacy of the therapeutic agent. This is overcome using a combination of an agent that has known therapeutic efficacy against the cancer to be treated by perturbation of the EGF signaling pathway and that stimulates expression of clusterin as a secondary effect, and an oligonucleotide that is effective to reduce the amount of clusterin in cancer cells. For example, the agent may be an antibody specific for HER-2, a small molecule inhibitor of HER-2, an antisense oligonucleotide specific for HER-2, or a peptide agent capable of interfering with HER-2 protein. The oligonucleotide may be an antisense oligonucleotide or an RNAi oligonucleotide.

This application claims priority from U.S. Provisional Applications 60/522,948 filed Nov. 23, 2004 and 60/522,960 filed Nov. 24, 2004, both of which are incorporated herein by reference.

### FIELD OF THE INVENTION

The present application relates to a method for treating cancer in a mammalian subject using a combination of therapeutic agents, one of which is an oligonucleotide effective to reduce the amount of clusterin, also known as testosterone-repressed prostate message-2 (TRPM-2) in the cancer cells, and the other of which is an agent that perturbs the EGF cell signaling pathway, and also stimulates the expression of clusterin as a consequence of its action on the target. Examples of agents that perturb the EGF signaling pathway include agents that target HER-2.

### BACKGROUND OF THE INVENTION

After lung cancer, breast cancer is the second leading cause of cancer deaths in women. According to the World Health Organization, more than 1.2 million people will be diagnosed with breast cancer this year worldwide, and The American Cancer Society estimates that in 2004, over 200,000 women in the United States will be diagnosed with invasive breast cancer (Stages I-IV), and about 40,000 women and almost 500 men will die from breast cancer in the United States in 2004.

The incidence rate of breast cancer (number of new breast cancers per 100,000 women) increased by approximately 4% during the 1980s but leveled off to 100.6 cases per 100,000 women in the 1990s.

Standard treatments include surgery, radiation, chemotherapy and hormonal therapies. Each of these treatments has drawbacks including loss of breast tissue, illness associated with radiation or chemotherapy, reproductive and hormonal side effects, and unreliable survival rates.

Thus breast cancer is a serious disease, fatal in many cases, and requires improved treatments to reduce fatalities and prevalence.

Clusterin or “TRPM-2” is a ubiquitous protein, with a diverse range of proposed activities. In prostate epithelial cells, expression of clusterin increases immediately following castration, reaching peak levels in rat prostate cells at 3 to 4 days post castration, coincident with the onset of massive cell death. These results have led some researchers to the conclusion that clusterin is a marker for cell death, and a promoter of apoptosis. On the other hand, Sertoli cells and some epithelial cells express high levels of clusterin without increased levels of cell death. Sensibar et al., (1995)[1] reported on in vitro experiments performed to more clearly elucidate the role of clusterin in prostatic cell death. The authors used LNCaP cells transfected with a gene encoding clusterin, and observed whether expression of this protein altered the effects of tumor necrosis factor α (TNFα), to which LNCaP cells are very sensitive. Treatment of the transfected LNCaP cells with TNFα resulted in a transient increase in clusterin levels for a few hours, but these levels had dropped by the time DNA fragmentation preceding cell death was observed.

United States published patent application US 20030166591 discloses the use of antisense therapy which reduces the expression of clusterin for the treatment of cancer of prostate and renal cell cancer.

U.S. Pat. No. 6,383,808 discloses compositions, particularly oligonucleotides, and methods for modulating the expression of clusterin.

United States published patent application 2004096882 discloses RNAi therapeutic probes targeting cancer associated proteins including clusterin.

United States published patent application US2004053874 discloses antisense modulation of clusterin expression.

United States published patent application US 2003166591 discloses clusterin antisense therapy using an oligonucleotide having 2′-O-(2-methoxy)ethyl modifications.

United States published patent application US 2003158130 discloses the use of chemotherapy-sensitization and radiation-sensitization of cancer by antisense clusterin oligodeoxynucleotides.

### SUMMARY OF THE INVENTION

Applicants have found that agents that perturb the EGF signaling pathway and that are known to be useful in the treatment of cancer can result in increased expression of the protein clusterin. Since clusterin can provide protection against apoptosis, this secondary effect detracts from the efficacy of the therapeutic agent. In overcoming this, the present invention provides a combination of therapeutic agents that is useful in the treatment of cancer. The combination comprises (i) an agent that has known therapeutic efficacy against the cancer to be treated and that perturbs the EGF signaling pathway and stimulates expression of clusterin as a secondary effect, and (ii) an oligonucleotide that is effective to reduce the amount of clusterin in cancer cells. In some embodiments of the invention, the agent with known therapeutic efficacy against the cancer is one that perturbs the EGF cell signaling pathway is an agent that interacts with HER-2. For example, the agent may be an antibody specific for HER-2, a small molecule inhibitor of HER-2, an antisense oligonucleotide specific for HER-2, or a peptide agent capable of interfering with HER-2 protein. The oligonucleotide may be an antisense oligonucleotide or an RNAi oligonucleotide.

The combination of the invention is useful in a method for treating cancer in a mammalian subject, comprising administering to the subject the known therapeutic agent and an oligonucleotide effective to reduce the amount of clusterin in the cancer cells.

The cancer may be breast cancer, osteosarcoma, lung cancer, pancreatic cancer, salivary gland cancer, colon cancer, prostate cancer, endometrial cancer, and bladder, for example.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1A shows a cytofluorimetric analysis of HER-2 protein expression in BT474 cells untreated (gray area), and treated with 10 (thin line), 25 μg/ml (thick line) of trastuzumab for 48 h, and negative control (dotted area);

FIG. 1B shows the number of adherent cells (black columns) and percentage of apoptotic cells (white columns) in BT474 cells, untreated and treated with 10 and 25 μg/ml of trastuzumab for 48 h;

FIG. 2 shows data for cells treated with 500 nM of either clusterin ASO or mismatch (MM) control oligodeoxynucleotide for 6 h, followed by exposure to 25 μg/ml of trastuzumab or control medium, 48 h after treatment;

FIG. 3 is a histogram showing the relative percentages of cells in the different phases of cell cycle (measured using propidium iodide staining and flow cytometry) after treatment with 500 nM of either clusterin ASO or MM control oligonucleotide for 6 h followed by exposure to 25 μg/ml of trastuzumab or control medium;

FIG. 4 shows the cytofluorimetric analysis of annexin V/PI staining for cells that were treated with 500 nM of either clusterin ASO or MM control oligonucleotide for 6 h, followed by exposure to 25 μg/ml of trastuzumab or control medium. Annexin V-positive cells are highlighted in the box; and

### DETAILED DESCRIPTION OF THE INVENTION

### Definitions

As used in the specification and claims of this application, the term “EGF cell signaling pathway” refers to the intracellular pathway that is stimulated upon binding of a ligand to a member of the epidermal growth factor receptor (EGFr) family. The EGFr family comprises EGFR, HER-2, HER-3 and HER-4. The EGFr family lies at the beginning of a complex signal transduction/communication pathway that modulates cell proliferation, survival, migration and differentiation.

As used in the specification and claims of this application, the phrase “an agent that perturbs the EGF cell signaling pathway” refers to any agent which is capable of disrupting the EGF cell signal, and includes an antibody specific for any of the members of the EGFr family, a small molecule inhibitor of normal binding to any member of the EGFr family, an antisense oligonucleotide that specifically inhibits expression of a member of the EGFr family, or a peptide agent capable of interfering with the signaling function of a member of the EGFr family.

As used in the specification and claims of this application, the term “clusterin” refers to the glycoprotein originally derived from ram rete testes, and to homologous proteins derived from other mammalian species, including humans, whether denominated as clusterin or an alternative name. The sequences of numerous clusterin species are known. For example, the sequence of human clusterin is reported by Wong et al., (1994) [2], and in NCBI sequence accession number NM_{—}001831 and is set forth in Seq. ID No. 1. In this sequence, the coding sequence spans bases 48 to 1397.

As used in this application, the term “amount of clusterin” refers to the amount of clusterin which is present in a form which is functional to provide anti-apoptotic protection. The effective amount of clusterin may be reduced through restricting production of clusterin (at the transcription or translation level) or by degrading clusterin at a rate faster than it is being produced. Further, it will be appreciated that inhibition occurs when the clusterin would otherwise be present if the antisense oligonucleotide had not been administered.

As used in the specification, “antisense oligonucleotide” refers to stretches of single-stranded DNA, usually chemically modified, whose sequence (3′→5′) is complementary to the sense sequence of a molecule of mRNA. Antisense molecules thereby effectively inhibit gene expression by forming RNA/DNA duplexes, and offer a more targeted option for cancer therapy than chemotherapy or radiation. Antisense is believed work by a variety of mechanisms, including physically blocking the ability of ribosomes to move along the messenger RNA, and hastening the rate at which the mRNA is degraded within the cytosol. The abbreviation ASO may also be used to refer to an antisense oligonucleotide.

As used in the specification and claims of this application, the term “combination” refers to an assemblage of reagents for use in therapy either by simultaneous or contemporaneous administration. Simultaneous administration refers to administration of an admixture (whether a true mixture, a suspension, an emulsion or other physical combination) of the agent that perturbs the EGF cell signaling pathway and the oligonucleotide. In this case, the combination may be the admixture or separate containers of the agent and the oligonucleotide that are combined just prior to administration. Contemporaneous administration refers to the separate administration of the agent and the oligonucleotide at the same time, or at times sufficiently close together that a synergistic activity relative to the activity of either the agent or the oligonucleotide alone is observed. In this, the combination comprises separate containers of the agent and the oligonucleotide

Agents that Perturb the EGF Cell Signaling Pathway

As noted above, in one embodiment, the present invention makes use of an agent that perturbs the EGF cell signaling pathway. This agent can be any agent which is capable of disrupting the EGF cell signal, and includes an antibody specific for any of the members of the EGFr family, a small molecule inhibitor of normal binding to any member of the EGFr family, an antisense oligonucleotide that specifically inhibits expression of a member of the EGFr family, or a peptide agent capable of interfering with the signaling function of a member of the EGFr family.

In some embodiments of the invention, the agent is one that perturbs the EGF signalling pathway by interaction with HER-2. HER-2, also known as ERBB2, or human epidermal growth factor receptor 2, helps control how cells grow, divide, and repair themselves. There has been extensive research done on the HER-2 gene and the role of the HER-2 protein in cancer dating from the 1980s. The HER-2 gene directs the production of special proteins, called HER-2 receptors. HER-2 is overexpressed in about a third of all breast cancers and is the target of trastuzumab.

One type of agent that can be used to interact with the HER-2 receptor and perturb the EGF signaling pathway is a pharmaceutical monoclonal antibody. The antibody may be specific for, and bind to, the HER-2 receptor. Alternately the antibody may bind to a related receptor and affect the HER-2 pathway in that way. This antibody may be trastuzumab, which is believed to block the HER-2 receptors when there is overexpression, and thereby block tumor growth and development. Trastuzumab, sold under the brand name Herceptin™, is a recombinant monoclonal antibody administered intravenously to treat breast cancer. Trastuzumab is currently used in combination with paclitaxel and is indicated for treatment of patients with metastatic breast cancer whose tumors over-express the HER-2 protein.

Other agents capable of perturbing the HER-2 cell signaling pathway include antisense agents capable of blocking HER-2 expression. US Patent publication 2003105051 discloses nucleic acid therapeutics for conditions related to levels of HER-2, and U.S. Pat. Nos. 5,910,583 and 6,365,345 disclose antisense nucleic acids for the prevention and treatment of disorders in which expression of c-erbB or erbB2 play a role

The examples below demonstrate that increased clusterin expression is observed when trastuzumab is used to target HER-2 in breast cancer cells. The extension of this observation to the EGF signaling pathway generally is reasonable, particularly in view of the fact that prior work has shown the reverse effect, i.e., that stimulation of EGF pathways, for example in renal cells, results in the inhibition of clusterin expression.

Small molecules capable of acting as the agent to interact with HER-2 and perturb EGF cell signaling include, for example, those disclosed in published patent documents U.S. Pat. No. 5,721,237, WO 03035843, and EP1131304. Peptides and peptide mimetics capable of acting as the agent to interact with HER-2 receptor and perturb the EGF signaling pathway include those such as adamanolol and disclosed in, for example, published patent documents CA2373721 and US2004006106.

As an alternative to agents that interact with HER-2, agents that interact with other members of the EGFr family may also be employed in the invention. For example, Erbitux™ (cetuximab) is a known pharmaceutical monoclonal antibody that targets the EGF cell signalling pathways via EGFr in colon and head and neck carcinoma.

### Oligonucleotides

Antisense Oligonucleotides (ASO)

Antisense oligonucleotides are synthetic polymers made up of monomers of deoxynucleotides like those in DNA. In the present application, the term antisense oligonucleotides includes antisense oligodeoxynucleotides.

The antisense oligonucleotides for use in the combination and method of the invention for treatment of cancer in humans are complementary to the nucleotide sequence of human clusterin as set forth in Seq. ID No. 1. In specific embodiments, the antisense oligonucleotide may span either the translation initiation site or the termination site of clusterin. The antisense oligonucleotide comprises and may consist essentially of an oligonucleotide selected from the group consisting of Seq. ID. Nos.: 2 to 19, or more specifically Seq. ID. No. 4, Seq. ID. No. 5 and Seq. ID. No. 12. As used in the specification and claims of this application, the phrase “consist essentially of” means that the oligonucleotide contains just the bases of the identified sequence or such bases and a small number of additional bases that do not materially alter the antisense function of the oligonucleotide In order avoid digestion by DNAse, antisense oligonucleotides and oligonucleotides are often chemically modified. For example, phosphorothioate oligodeoxynucleotides are stabilized to resist nuclease digestion by substituting one of the non-bridging phosphoryl oxygen of DNA with a sulfur. Increased antisense oligonucleotide stability can also be achieved using molecules with 2-methoxyethyl (MOE) substituted backbones as described generally in U.S. Pat. No. 6,451,991, incorporated by reference in those jurisdictions allowing such incorporation, and US published patent application US-2003-0158143-A1. Thus, in the combination and method of the invention, the antisense oligonucleotide may be modified to enhance in vivo stability relative to an unmodified oligonucleotide of the same sequence. The modification may be a (2′-O-(2-methoxyethyl)) modification. The oligonucleotide may have a phosphorothioate backbone throughout, the sugar moieties of nucleotides 1-4 and 18-21 may bear 2′-O-methoxyethyl modifications and the remaining nucleotides may be 2′-deoxynucleotides.

The antisense oligonucleotide may be a 5-10-5 gap-mer methoxyl ethyl modified (MOE) oligonucleotide corresponding to SEQ ID NO.: 5 below. The antisense oligonucleotide may be from 10-25 bases in length, or from 15-23 bases in length, or from 18-22 bases in length, or 21 bases in length.

Exemplary sequences which can be employed as antisense oligonucleotides in the combination and method of the invention are disclosed in PCT Patent Publication WO 00/49937, US Patent Publication US-2002-0128220-A1, and U.S. Pat. No. 6,383,808, all of which are incorporated herein by reference in those jurisdictions where such incorporation is permitted. Specific antisense oligonucleotide sequences are set forth in the present application as Seq. ID Nos.: 2 to 19 and are represented in Table 1.

TABLE 1

| Seq ID | | |
|---|---|---|
| No. | Description | SEQUENCE (5′ to 3′) |
| 2 | Antisense TRPM-2 | GCACAGCAGGAGAATCTTCAT |
| | oligonucleotide | |
| 3 | Antisense TRPM-2 | TGGAGTCTTTGCACGCCTCGG |
| | oligonucleotide | |
| 4 | Antisense | CAGCAGCAGAGTCTTCATCAT |
| | oligonucleotide | |
| | corresponding to | |
| | the human TRPM-2 | |
| | translation | |
| | initiation site | |
| 5 | Antisense TRPM-2 | ATTGTCTGAGACCGTCTGGTC |
| | oligonucleotide | |
| 6 | Antisense TRPM-2 | CCTTCAGCTTTGTCTCTGATT |
| | oligonucleotide | |
| 7 | Antisense TRPM-2 | AGCAGGGAGTCGATGCGGTCA |
| | oligonucleotide | |
| 8 | Antisense TRPM-2 | ATCAAGCTGCGGACGATGCGG |
| | oligonucleotide | |
| 9 | Antisense TRPM-2 | GCAGGCAGCCCGTGGAGTTGT |
| | oligonucleotide | |
| 10 | Antisense TRPM-2 | TTCAGCTGCTCCAGCAAGGAG |
| | oligonucleotide | |
| 11 | Antisense TRPM-2 | AATTTAGGGTTCTTCCTGGAG |
| | oligonucleotide | |
| 12 | Antisense TRPM-2 | GCTGGGCGGAGTTGGGGGCCT |
| | oligonucleotide | |
| 13 | Antisense TRPM-2 | GGTGTAGACG CCGCACG |
| | oligonucleotide | |
| 14 | Antisense TRPM-2 | GCAGCGCAGC CCCTGG |
| | oligonucleotide | |
| 15 | Antisense TRPM-2 | GCAGCAGCCG CAGCCCGGCT CC |
| | oligonucleotide | |
| 16 | Antisense TRPM-2 | AGCCGCAGCC CGGCTCCT |
| | oligonucleotide | |
| 17 | Antisense TRPM-2 | CAGCAGCCGC AGCCCGGCTC |
| | oligonucleotide | |
| 18 | Antisense TRPM-2 | GCAGCAGCCG CAGCCCGGCT |
| | oligonucleotide | |
| 19 | Antisense TRPM-2 | AGCAGCCGCAGCCCGGCTCC |
| | oligonucleotide | |
| 20 | 2 base TRPM-2 | CAGCAGCAGAGTATTTATCAT |
| | mismatch | |
| | oligonucleotide | |
| | used as a control | |

A particularly preferred antisense oligonucleotide is a 21 mer oligonucleotide (CAGCAGCAGAGTCTTCATCAT; SEQ ID NO.: 4) targeted to the translation initiation codon and next 6 codons of the human clusterin sequence with a 2′-MOE modification. In one embodiment, this oligonucleotide has a phosphorothioate backbone throughout. The sugar moieties of nucleotides 1-4 and 18-21 (the “wings”) bear 2′-O-methoxyethyl modifications and the remaining nucleotides (nucleotides 5-17; the “deoxy gap”) are 2′-deoxynucleotides. Cytosines in the wings (i.e., nucleotides 1, 4 and 19) are 5-methylcytosines.

RNAi Oligonucleotides

Reduction in the amount of clusterin may also be achieved using RNA interference or “RNAi”. RNAi is a term initially coined by Fire and co-workers to describe the observation that double-stranded RNA (dsRNA) can block gene expression[3]. Double stranded RNA, or dsRNA directs gene-specific, post-transcriptional silencing in many organisms, including vertebrates. RNAi involves mRNA degradation, but many of the biochemical mechanisms underlying this interference are unknown. The use of RNAi has been further described[3,4].

The initial agent for RNAi is a double stranded RNA molecule corresponding to a target nucleic acid. The dsRNA is then thought to be cleaved in vivo into short interfering RNAs (siRNAs) which are 21-23 nucleotides in length (19-21 bp duplexes, each with 2 nucleotide 3′ overhangs). Alternatively, RNAi may be effected via directly introducing into the cell, or generating within the cell by introducing into the cell a suitable precursor (e.g. vector, etc.) of such an siRNA or siRNA-like molecule. An siRNA may then associate with other intracellular components to form an RNA-induced silencing complex (RISC).

RNA molecules used in embodiments of the present invention generally comprise an RNA portion and some additional portion, for example a deoxyribonucleotide portion. The total number of nucleotides in the RNA molecule is suitably less than 49 in order to be effective mediators of RNAi. In preferred RNA molecules, the number of nucleotides is 16 to 29, more preferably 18 to 23, and most preferably 21-23.

In certain embodiments of the invention, the siRNA or siRNA-like molecule is less than about 30 nucleotides in length. In a further embodiment, the siRNA or siRNA-like molecules are about 21-23 nucleotides in length. In an embodiment, siRNA or siRNA-like molecules comprise and 19-21 bp duplex portion, each strand having a 2 nucleotide 3′ overhang.

In certain embodiments of the invention, the siRNA or siRNA-like molecule is substantially identical to a clusterin-encoding nucleic acid or a fragment or variant (or a fragment of a variant) thereof. Such a variant is capable of encoding a protein having clusterin-like activity. In some embodiments, the sense strand of the siRNA or siRNA-like molecule being to the same target region as to the antisense species of SEQ ID NO: 4 or a fragment thereof (RNA having U in place of T residues of the DNA sequence). In other embodiments, the RNAi sequence consists of Seq. Id. No. 41 or 43. For example, United States published patent application 2004096882 discloses RNAi therapeutic probes targeting clusterin. In addition, reagents and kits for performing RNAi are available commercially from for example Ambion Inc. (Austin, Tex., USA) and New England Biolabs Inc. (Beverly, Mass., USA). Suitable sequences for use as RNAi in the present invention are set forth in the present application as Seq. ID Nos. 21 to 44 as shown in Table 2.

TABLE 2

| SEQ ID | | |
|---|---|---|
| No. | Description | SEQUENCE |
| 21 | RNAi for human | GUAGAAGGGC GAGCUCUGGTT |
| | clusterin | |
| 22 | RNAi for human | GAUGCUCAACACCUCCUCCT T |
| | clusterin | |
| 23 | RNAi for human | GGAGGAGGUG UUGAGCAUCT T |
| | clusterin | |
| 24 | RNAi for human | CUAAUUCAAU AAAACUGUCT T |
| | clusterin | |
| 25 | RNAi for human | GACAGUUUUA UUGAAUUAGT T |
| | clusterin | |
| 26 | RNAi for human | UAAUUCAACA AAACUGUTT |
| | clusterin | |
| 27 | RNAi for human | ACAGUUUUGU UGAAUUATT |
| | clusterin | |
| 28 | RNAi for human | AUGAUGAAGA CUCUGCUGCT T |
| | clusterin | |
| 29 | RNAi for human | GCAGCAGAGU CUUCAUCAUT T |
| | clusterin | |
| 30 | RNAi for human | UGAAUGAAGG GACUAACCUG TT |
| | clusterin | |
| 31 | RNAi for human | CAGGUUAGUC CCUUCAUUCA TT |
| | clusterin | |
| 32 | RNAi for human | CAGAAAUAGA CAAAGUGGGG TT |
| | clusterin | |
| 33 | RNAi for human | CCCCACUUUG UCUAUUUCUG TT |
| | clusterin | |
| 34 | RNAi for human | ACAGAGACUA AGGGACCAGA TT |
| | clusterin | |
| 35 | RNAi for human | ACAGAGACUA AGGGACCAGA TT |
| | clusterin | |
| 36 | RNAi for human | CCAGAGCUCG CCCUUCUACT T |
| | clusterin | |
| 37 | RNAi for human | GUAGAAGGGC GAGCUCUGGT T |
| | clusterin | |
| 38 | RNAi for human | GUCCCGCAUC GUCCGCAGCT T |
| | clusterin | |
| 39 | RNAi for human | GCUGCGGACG AUGCGGGACT T |
| | clusterin | |
| 40 | RNAi for human | CUAAUUCAAU AAAACUGUCT T |
| | clusterin | |
| 41 | RNAi for human | GACAGUUUUA UUGAAUUAGT T |
| | clusterin | |
| 42 | RNAi for human | AUGAUGAAGA CUCUGCUGC |
| | clusterin | |
| 43 | RNAi for human | GCAGCAGAGU CUUCAUCAU |
| | clusterin | |
| 44 | RNAi for human | CCAGAGCUCG CCCUUCUACT T |
| | clusterin | |

Cancers that can be Treated

The combination of the present application is useful in the treatment of a variety of cancers in which EGFr inhibition is significant. These cancers include breast cancer, osteosarcoma, lung cancer, pancreatic cancer, salivary gland cancer, colon cancer, prostate cancer, endometrial cancer, and bladder cancer.

A variety of reagents targeting the EGFr family have been tested for efficacy in the treatment of lung cancer. These reports are summarized in Tiseo (2004) [9]

Targeting of Her2/neu expression has been shown to have therapeutic potential in controlling the development and progression of prostate cancer. Di Lorenzo (2004) [10] Her-2/neu has also been shown to be a target in ovarian cancer, Xu (2003) [11]; salivary gland cancer, Scholl (2001)[12]; endometrial cancer Cianciulli (2003)[13] and Slomovitz (2004)[14]; pancreatic cancer Baxevanis (2004)[15]; colon and colorectal cancer, Park (2004)[16], Half (2004) [17] and Nathanson (2003)[18]; and bladder cancer, Bellmunt (2003)[19].

Methods

Administration of antisense oligonucleotides can be carried out using the various mechanisms known in the art, including naked administration and administration in pharmaceutically acceptable lipid carriers. For example, lipid carriers for antisense delivery are disclosed in U.S. Pat. Nos. 5,855,911 and 5,417,978. In general, the antisense is administered by intravenous, intraperitoneal, subcutaneous or oral routes, or direct local tumor injection.

The amount of antisense oligonucleotide administered is one effective to reduce the expression of clusterin in cancer cells, particularly and surprisingly when in combination with an agent that perturbs the HER-2 cell signaling pathway. It will be appreciated that this amount will vary both with the effectiveness of the antisense oligonucleotide employed, and with the nature of any carrier used. The determination of appropriate amounts for any given composition is within the skill in the art, through standard series of tests designed to assess appropriate therapeutic levels. In one embodiment, the antisense oligonucleotide is administered to a human patient in an amount of between 40-640 mg, or more particularly, from 300-640 mg. In another embodiment, the antisense oligonucleotide is administered according to the weight of the subject in need of the treatment. For example, the antisense oligonucleotide may be provided at a dosage of from 1 to 20 mg/kg of body weight.

The monoclonal trastuzumab is available as a powder in a vial containing 440 mg of drug. It must be mixed with a liquid before intravenous injection, often with an initial dose of 4 mg per kilogram of body weight followed by a weekly dose of 2 mg per kilogram of body weight. See, for example, Slamon, D J et al., 2001 [5]

Additional Therapeutic Agents

The method for treating cancer in accordance with one embodiment of the invention may further include administration of chemotherapy agents or other agents useful in breast cancer therapy and/or additional antisense oligonucleotides directed at different targets in combination with the therapeutic effective to reduce the amount of active clusterin. For example, antisense clusterin oligonucleotide may be used in combination with more conventional chemotherapy agents such as taxanes (paclitaxel or docetaxel), mitoxanthrone, doxorubicin, gemcitabine, cyclophosphamide, decarbazine, topoisomerase inhibitors), angiogenesis inhibitors, differentiation agents and signal transduction inhibitors.

The application is further described in the following non-limiting examples.

### EXAMPLES

### Materials and Methods

Tumour Cells

The BT474 human breast carcinoma cell line was cultured in DMEM supplemented with 10% fetal calf serum, glutamine, penicillin and streptomycin sulfate at 37° C. under 5% CO₂-95% air. Cell culture reagents were purchased from Invitrogen (Milan, Italy).

Reagents

Trastuzumab (Herceptin®, purchased from Roche, Monza, Italy) was stored at 4° C. and adjusted to the final concentration with culture medium.

Phosphorothioate oligonucleotides used in this study were purchased from La Jolla Pharmaceuticals Co. (La Jolla, Calif., USA) or provided by OncoGenex Technologies Inc., Vancouver, Canada. The sequence of the clusterin ASO used corresponded to the human clusterin translation initiation site (5′-CAGCAGCAGAGTCTTCATCAT-3′) (SEQ ID NO.: 4). A 2-base clusterin mismatch oligonucleotide (5′-CAGCAGCAGAGTATTTATCAT-3′) (SEQ ID NO.: 20) was used as control. Oligonucleotides were delivered into cells in form of complexes with the Lipofectin™ transfection reagent (Invitrogen). Cells were incubated with different concentrations of oligonucleotides and Lipofectin™ for 6 hours in OPTIMEM™ medium (Gibco). At the end of oligonucleotide treatment, the medium was replaced with fresh growth medium containing 2% of fetal calf serum and at different time points, cells were processed according to the various analyses to be performed.

Proliferation Assay

5×10⁵ cells were seeded in 60-mm culture dishes (Nunc™, Mascia Brunelli, Milano, Italy) and allowed to attach. Forty-eight hours after seeding, cells were the treated with clusterin ASO at 500 nM for 6 h. After oligonucleotide treatment, cells were exposed to a 25 μM concentration of trastuzumab. At different time points during treatment, cells were harvested and assessed using a Cell Viability Analyzer™ (Coulter, Model Vi-Cell XR, Beckman, Fla., USA).

Western Blot

Western blot and detection were performed as previously reported, for example in [7]. Briefly, 40 μg of total proteins were loaded on denaturing SDS-PAGE. Immunodetection of clusterin and PARP proteins was performed by using, respectively, mouse anti-clusterin (1:1000, 41 D, Upstate Biotechnology, NY, USA) and rabbit anti-PARP (1:2000, VIC5, Boehringer Mannheim, Germany). To check the amount of proteins transferred to nitrocellulose membrane, HSP 72/73 was used as control and detected by an anti-human HSP 72/73 (1:1000, Calbiochem Cambridge, Mass., USA). The relative amounts of the transferred proteins were quantified by scanning the autoradiographic films with a gel densitometer scanner (Bio-Rad, Milano, Italy) and normalized to the related HSP72/73 amounts.

Cell Cycle Analysis

Measuring the percentage of cells in different phases of cell cycle was performed by flow cytometry (Becton-Dickinson, Heidelberg, Germany) as previously described Telford, W. G., L. E. King, and P. J. Fraker (1992) Comparative evaluation of several DNA binding dyes in the detection of apoptosis-associated chromatin degradation by flow cytometry. Cytometry. 13: 137-143 [8]. Briefly, 2×10⁵ adherent cells were fixed and resuspended in a solution containing the dye propidium iodide (PI) at a concentration of 50 μg/ml. Percentages of cells in the different phases of the cell cycle were calculated using CELLQuest™ software (Becton Dickinson).

Evaluation of Apoptosis

Apoptosis was detected by flow cytometric analysis of sub-G1 peaks, and also analyzed by Annexin V-FITC versus PI assay (Vybrant™ Apoptosis Assay, V-13242, Molecular Probes, Eugene, Oreg., USA). Briefly, adherent cells were harvested, suspended in the annexin-binding buffer (1×10⁶ cells/ml) and incubated with the annexin V-FITC and PI for 15 min, at room temperature in the dark, then immediately analyzed by flow-cytometry. The data are presented as bi-parametric dot plots showing the annexin V-FITC green fluorescence versus the PI red fluorescence.

### Example 1

Enhanced expression of clusterin after treatment with trastuzumab in BT474 HER-2 overexpressing breast cancer cells.

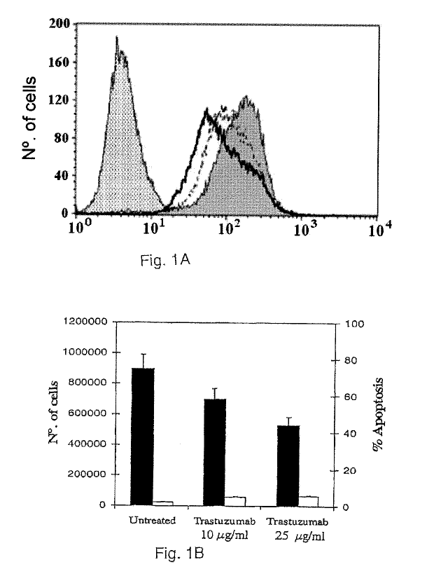

The human breast cancer cell line BT474, which overexpresses the HER-2 gene, was exposed to clinically relevant concentrations of trastuzumab. The treatment of BT474 cells with trastuzumab down-regulated HER-2 protein expression in a dose-dependent manner. Analysis by flow cytometry revealed that the mean channel of fluorescence decreased from 173 to 112 and 72 in the BT474 cells treated with 10 and 25 μg/ml of trastuzumab, respectively. FIG. 1A shows a cytofluorimetric analysis of HER-2 protein expression in BT474 cells untreated (gray area), and treated with 10 (thin line), 25 μg/ml (thick line) of trastuzumab for 48 h, and negative control (dotted area).

Trastuzumab-mediated reduction of HER-2 protein expression was accompanied by inhibition of BT474 cell growth without affecting apoptosis (FIG. 1B). The simultaneous analysis of number of viable and apoptotic cells demonstrated that the treatment with trastuzumab significantly decreased cell growth in a dose-dependent effect, since the number of cells was reduced from about 1×10⁶ to 8×10⁵ and 6×10⁵ in the BT474 cells treated with 10 and 25 μg/ml of trastuzumab, respectively. However, trastuzumab treatment even at the highest dose of 25 μg/ml induced little or no increase in the percentage of apoptotic cells (less than 10%).

It was next evaluated whether trastuzumab treatment was able to modulate clusterin/apolipoprotein J expression. Western blot analysis of clusterin protein in the lysates of BT474 cells exposed to trastuzumab (10-25 μg/ml) for up to 48 h. The relative amount of the transferred clusterin protein was quantified and normalized relative to the corresponding HSP 72/73 protein amount. A representative of three independent experiments with similar results was evaluated. Both trastuzumab concentrations upregulated clusterin protein expression. At 25 μg/ml trastuzumab an increase of 3 fold of the 60 kD form and the appearance of the 40 kD form was observed.

### Example 2

Effect of combined treatment with clusterin ASO and trastuzumab on BT474 cell growth rate.

The effect of treatment with oligonucleotides on clusterin protein expression was evaluated. BT474 cells were transfected with 300 or 500 nM clusterin ASO (Seq. ID No. 4 with MOE modifications) (or the same concentrations of its mismatch oligonucleotide as control, as described supra), and clusterin expression was analyzed by Western blot 48 h after treatment. Treatment of BT474 with 100 or 500 nM clusterin ASO for 6 h reduced clusterin protein expression by about 30 and 50% compared to mismatch control, respectively.

The analysis was performed 48 h after the end of treatment. The relative amount of the transferred clusterin protein was quantified and normalized to the corresponding HSP72/73 protein amount.

In contrast, clusterin levels were not affected by the 2-base mismatch (MM) control oligonucleotide at any of the used concentrations.

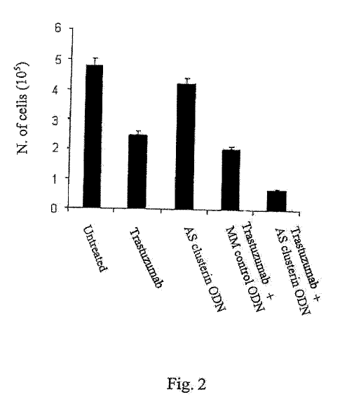

To determine whether treatment with clusterin ASO (Seq. ID No. 4, with MOE modification) enhances the cytotoxic effect of trastuzumab, BT474 cells were treated with 500 nM clusterin ASO or MM control oligonucleotide and then exposed to 25 μg/ml trastuzumab for 48 h. FIG. 2 shows the number of cells in the BT474 untreated and treated with trastuzumab alone and in combination with clusterin ASO or MM control oligonucleotide. Trastuzumab reduced the growth of BT474 by about 50%, while treatment with clusterin ASO did not show any effect on cell proliferation. The combination of trastuzumab with clusterin ASO, but not with the MM control oligonucleotide, significantly enhanced chemosensitivity of cells, with reduction of cell proliferation of up to 85%.

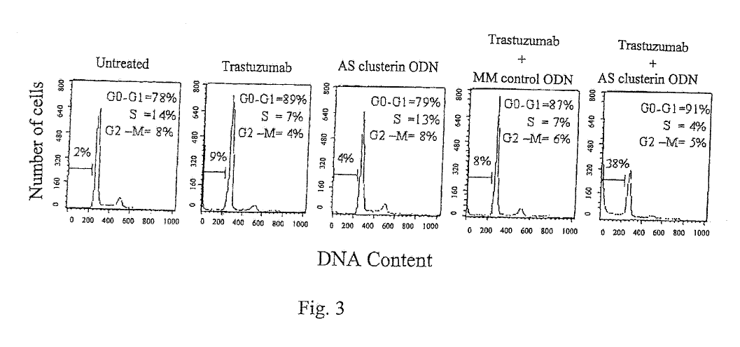

To study the mechanisms by which the treatment with clusterin ASO enhanced the chemosensitivity of cells to trastuzumab, cell cycle distribution was analyzed by flow cytometry. FIG. 3 shows the histograms of DNA content in BT474 cells both untreated and treated with the single agents alone or in combination. The analysis was performed 48 h after the end of treatments.

Analysis of cell percentages in the different phases of the cell cycle revealed that, while the treatment with clusterin ASO did not show any effect on cell cycle distribution, exposure of cells to trastuzumab induced a decrease of proliferative compartment (S-G₂/M) with the concomitant increase in the G₀/G₁ phase of the cell cycle. However, both treatments did not induce the appearance of populations with a sub-G1 DNA content. Cell cycle distribution of cells treated with trastuzumab plus MM control oligonucleotide was similar to that of trastuzumab alone. In contrast, a strong perturbation of the cell cycle was observed in the cells treated with trastuzumab and Clusterin ASO and a significant fraction of the cell population (about 40%) resided to the sub-G₁ compartment.

### Example 3

Effect of combined treatment with clusterin ASO and trastuzumab on the induction of apoptosis.

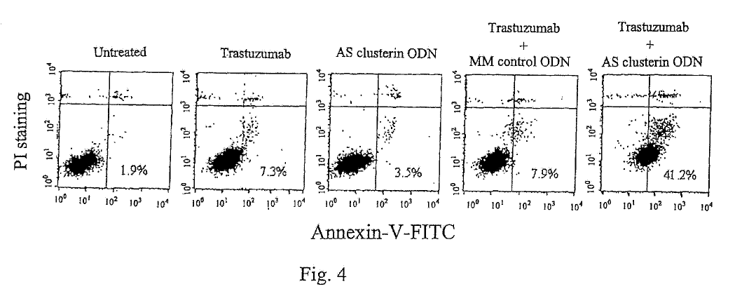

Apoptosis was evaluated in BT474 cells exposed to the different treatments. FIG. 4 shows the cytofluorimetric analysis of the annexin V versus PI staining performed in the BT474 untreated and treated with trastuzumab alone and in combination with Clusterin ASO or MM control oligonucleotide. Using the same treatment schedule described above, apoptosis (shown as the annexin V⁺/PI⁻ region of the dot plot panels) was observed only after combined treatment of Clusterin ASO plus trastuzumab. The percentage of annexin V⁺/PI⁻ cells was about 40% in the trastuzumab/clusterin combination and was less than 10% in all the other treatments.

### Example 4

### Effect on Clusterin Protein Expression

The effect of combined clusterin ASO and trastuzumab on clusterin protein expression and PARP cleavage was also evaluated. Cells were treated with 500 nM of either clusterin ASO or MM control oligonucleotide for 6 h, then exposed to 25 μg/ml of trastuzumab or control medium. Clusterin protein expression and poly (ADP-ribose) polymerase (PARP) cleavage were analyzed by Western blot. The relative amount of the transferred clusterin protein was quantified and normalized relative to the corresponding HSP 72/73 protein amount.

Expression of clusterin in the combination of trastuzumab plus MM control oligonucleotide is the same as that after treatment of trastuzumab alone, but the combination of trastuzumab with clusterin ASO blocked the trastuzumab-induced clusterin expression by specifically inhibiting its expression.

Analysis of the PARP cleavage demonstrated that the 116 KD intact form of PARP was observed in all samples examined, whereas the 85 KD PARP cleavage fragment was detected only after combined treatment with trastuzumab plus clusterin ASO.

All of the cited documents are incorporated herein by reference in those jurisdictions allowing such incorporation.

While specific embodiments of the invention have been described and illustrated, such embodiments should be considered illustrative of the invention only and not as limiting the invention.

### REFERENCES

1. Wong et al., Eur. J. Biochem. 221 (3), 917-925 (1994)
2. Sensibar et al., Cancer Research 55: 2431-2437 (1995)
3. Fire et al. (1998) Nature 391, 806-811.
4. Carthew et al. (2001) Current Opinions in Cell Biology 13, 244-248.
5. Slamon, D. J., Leyland-Jones, B., Shak, S., Fuchs, H., Paton, V., Bajamonde, A., Fleming, T., Eiermann, W., Wolter, J., Pegram, M., Baselga, J., Norton, L. (2001) NEJM Vol 344(11):783-792
6. Elbashir et al. (2001) Nature 411, 494-498.
7. Miyake et al. (2000) Testosterone-repressed prostate message-2 is an antiapoptotic gene involved in progression to androgen independence in prostate cancer. Cancer Res. Jan 1; 60(1):170-6.
8. Telford, W. G., L. E. King, and P. J. Fraker (1992) Comparative evaluation of several DNA binding dyes in the detection of apoptosis-associated chromatin degradation by flow cytometry. Cytometry. 13: 137-143.
9. Tiseo M., Loprevite, M. and Ardizzoni, A. (2004) Epidermal Growth Factor receptor inhibitorsL a new prospective in the treatment of lung cancer. Curr. Med. Chem. Anti-Canc. Agents 4: 139-48.
10. Di Lorenzo et al. (2004) HER-2/neu receptor in prostate cancer development and progression to androgen independence. Tumori 90: 163-70.
11. Xu, X. F. and Xing, P. X. (2003) Discovery and validation of new molecular targets for ovarian cancer. Curr. Opin. Mol. Ther. 5: 625-630.
12. Scholl, S. Beuzeboc, P, and Pouillart, P. (2001) Targeting Her-2 in other tumor types. Ann Oncol. 12 Suppl 1: S81-7.
13. Cianciulli et al. (2003. Her-2/neu oncogene amplification and chromosome 17 aneusomy in endometrial carcinoma: correlation with oncoprotein expression and conventional pathological parameters. J. Exp. Clin. Cancer Res. 22: 265-71.
14. Slomovitz et al., (2004). Her-2/neu overexpression and amplification in uterine papillary serous carcinoma. J. Clin. Oncol. 22: 3126-32.
15. Baxevanis et al. (2004) Immunobiology of HER-2/neu oncoprotein and its potential in cancer immunotherapy. Cancer Immunol. Immunother. 53: 166-75.
16. Park et al. (2004) Clinical significance of HER-2/neu expression in colon cancer. Korean J. Gastroenterol. 44: 147-52.
17. Half et al. (2004) HER-2 receptor expression, localization and activation in colorectal cancer cell lines and human tumors. Int. J. cancer 108: 540-8.
18. Nathanson et al. (2003) HER-2/neu expression and gene amplification in colon cancer. Int. J. cancer 105: 796-802.
19. Bellmunt, J, Hussain, M and Dinney, C. P. (2003). Novel approaches with targeted therapies in bladder cancer. Therapy of bladder cancer by blockade of the epidermal growth factor receptor family. Crit. Rev. Oncol. Hematol. 46 Suppl: S85-104.

1. A combination for treating cancer in a mammalian subject, comprising an agent that has a primary target other than clusterin and that is effective for treating cancer in that mammalian subject and that increases the expression level of clusterin and an oligonucleotide effective to reduce the effective amount of clusterin in the cancer cells, wherein the agent is an agent that perturbs EGF cell signaling pathways.

2. The combination of claim 1, wherein the agent that perturbs the EGF cell signaling pathway interacts with HER-2.

3. The combination of claim 2, wherein said agent is a monoclonal antibody specific for HER-2.

4. The combination of claim 3, wherein said agent is trastuzumab.

5. The combination of claim 1, wherein the oligonucleotide effective to reduce the amount of clusterin is an anti-clusterin antisense oligonucleotide.

6. The combination of claim 5, wherein said anti-clusterin antisense oligonucleotide spans either the translation initiation site or the termination site of clusterin.

7. The combination of claim 5 wherein said anti-clusterin antisense oligonucleotide is modified to enhance in vivo stability relative to an unmodified oligonucleotide of the same sequence.

8. The combination of claim 7, wherein said modification is a (2′-O-(2-methoxyethyl) modification.

9. The combination of claim 8, wherein said antisense oligonucleotide has a phosphorothioate backbone throughout, the sugar moieties of nucleotides 1-4 and 18-21, the “wings”, bear 2′-O-methoxyethyl modifications and the remaining nucleotides are 2′-deoxynucleotides.

10. The combination of claim 5, wherein said anti-clusterin antisense oligonucleotide consists essentially of an oligonucleotide selected from the group consisting of Seq. ID. Nos.: 2 to 19.

11. The combination of claim 5, wherein said anti-clusterin antisense oligonucleotide consists essentially of an oligonucleotide selected from the group consisting of Seq. ID. No. 4, Seq. ID. No. 5 and Seq. ID. No. 12.

12. The combination of claim 1, wherein the oligonucleotide effective to reduce the amount of clusterin is an RNAi oligonucleotide.

13. The combination of claim 12, wherein the RNAi oligonucleotide comprises a sequence selected from the group consisting of Seq. ID. Nos. 21-44.

14. The combination of claim 1, wherein the oligonucleotide effective to reduce the amount of clusterin and the agent are in a common pharmaceutically acceptable carrier or in separate pharmaceutically acceptable carriers.

15. (canceled)

16. The combination of claim 1, wherein the agent and the oligonucleotide effective to reduce the amount of clusterin are each provided in dosage unit form, either together or individually.

17. A method for treating cancer in a mammalian subject, comprising administering to the subject the combination of claim 1.

18. The method of claim 17, wherein said cancer is selected from the group consisting of breast cancer, osteosarcoma, lung cancer, pancreatic cancer, salivary gland cancer, colon cancer, prostate cancer, endometrial cancer, and bladder cancer.

19. The method of claim 18, wherein said cancer is breast cancer.

20-22. (canceled)

23. A combination for treating cancer in a mammalian subject, comprising an agent that has a primary target other than clusterin and that is effective for treating cancer in that mammalian subject and that increases the expression level of clusterin, and an oligonucleotide effective to reduce the effective amount of clusterin in the cancer cells, wherein the agent is perturbs the EGF cell signaling pathways, wherein the oligonucleotide and the agent are in a common pharmaceutically acceptable carrier or are administered contemporaneously.

24. A method for treating cancer in a mammalian subject, comprising administering to a subject in need of a combination of therapeutic agents comprising an oligonucleotide effective to reduce the amount of clusterin in the cancer cells, and an agent that perturbs the EGF cell signaling pathways and also stimulates the expression of clusterin in the cancer cells, wherein the oligonucleotide and the agent are in a common pharmaceutically acceptable carrier or are administered contemporaneously.
